(19) United States
(12) Patent Application Publication
(10) Pub. No.: US 2008/0260717 A1
(43) Pub. Date: Oct. 23, 2008

(54) Methods for Reducing Seizure-Induced Neuronal Damage

(75) Inventors: Shi Du Yan, Tenafly, NJ (US); Guy McKhann, Bronxville, NY (US); David M. Stern, Cincinnati, OH (US)

Correspondence Address: COOPER & DUNHAM, LLP, 1185 AVENUE OF THE AMERICAS, NEW YORK, NY, 10036, US

(73) Assignee: TRUSTEES OF COLUMBIA UNIVERSITY IN THE CITY OF NEW YORK, New York, NY (US)

(21) Appl. No.: 10/577,382

(22) PCT Filed: Oct. 28, 2004
(86) PCT No.: PCT/US2004/036173

§ 371 Date: Feb. 11, 2008

Related U.S. Application Data
(60) Provisional application No. 60/516,323, filed on Oct. 31, 2003.

(51) Int. Cl. A61K 39/395; A61K 31/7052; A61P 25/00

(52) U.S. Cl. 424/130.1; 514/44

(57) ABSTRACT

This invention provides a method for treating a subject either during or soon after a seizure, in order to reduce the extent of neuronal damage in the subject resulting from the seizure comprising administering to the subject, either during or soon after the seizure, a therapeutically effective amount of an inhibitor of receptor for advanced glycation endproducts (RAGE), so as to thereby reduce the extent of neuronal damage in the subject. This invention further provides a method for inhibiting neuronal damage which would otherwise result from a seizure in a subject predisposed to having a seizure, comprising administering to the subject a prophylactically effective amount of an inhibitor of receptor for advanced glycation endproducts (RAGE), so as to inhibit neuronal damage which would otherwise result from a seizure in the event the subject were to suffer a seizure.

This application claims priority of U.S. Provisional Application No. 60/516,323, filed Oct. 31, 2003, the contents of which are hereby incorporated by reference.

Throughout the application, various publications are referenced. Full citations for these publications may be found immediately preceding the claims. The disclosures of these publications are hereby incorporated by reference into this application in order to more fully describe the state of the art as of the date of the invention described and claimed herein.

### BACKGROUND OF THE INVENTION

Seizures

Human seizure disorders are a substantial health problem because of the large number of affected individuals and the variety of different syndromes. For example, an estimated 1% of the U.S. population is affected by over 40 different syndromes that make up the epilepsies (1, 2). All individuals are potentially vulnerable to seizures; they can occur in anyone following a sufficiently intense insult to the brain (3). Although seizures can occur in most anyone, individuals vary in what constitutes a seizure-inducing stimulus (4, 5). Some individuals have high seizure susceptibility such that they suffer spontaneous seizures while others have low susceptibility such that even head trauma or certain brain tumors would not lead to seizures (4).

Receptor for Advanced Glycation Endproducts (RAGE)

Receptor for Advanced Glycation Endproduct (RAGE) is a member of the immunoglobulin superfamily of cell surface molecules first discovered because of its interaction with products of nonenzymatic glycoxidation termed Advanced Glycation Endproducts (AGEs) (6). Subsequently, two endogenous ligands of RAGE have been identified, members of the S100/calgranulin family and the high mobility group I-type polypeptide amphoterin (7, 8). Whereas amphoterin appears to be expressed at high levels in tumors and during development (8-10), S100/calgranulins in the extracellular space are well-known for their association with inflammatory disorders; they have been found in colitis, arthritis, cystic fibrosis, and chronic bronchitis (11). RAGE has been identified as a central signal transduction receptor mediating effects of S100/calgranulins on key cellular targets, including mononuclear phagocytes (MPs), lymphocytes and vascular endothelium (7). The potential physiologic significance of this interaction was emphasized by inhibition of the delayed-type hypersensitivity response by blockade of RAGE-S100/calgranulin interaction (7).

### SUMMARY OF THE INVENTION

This invention provides a method for treating a subject either during or soon after a seizure, in order to reduce the extent of neuronal damage in the subject resulting from the seizure comprising administering to the subject, either during or soon after the seizure, a therapeutically effective amount of an inhibitor of receptor for advanced glycation endproducts (RAGE), so as to thereby reduce the extent of neuronal damage in the subject.

This invention further provides a method for inhibiting neuronal damage which would otherwise result from a seizure in a subject predisposed to having a seizure, comprising administering to the subject a prophylactically effective amount of an inhibitor of receptor for advanced glycation endproducts (RAGE), so as to inhibit neuronal damage which would otherwise result from a seizure in the event the subject were to suffer a seizure.

This invention further provides an article of manufacture comprising (a) a packaging material having therein an inhibitor of receptor for advanced glycation endproducts (RAGE) and (b) instructions for using the inhibitor to treat a subject during or soon after a seizure, in order to reduce the extent of neuronal damage in the subject resulting from the seizure.

Finally, this invention provides an article of manufacture comprising (a) a packaging material having therein an inhibitor of receptor for advanced glycation endproducts (RAGE) and (b) instructions for using the inhibitor to inhibit neuronal damage which would otherwise result from a seizure in a subject predisposed to having a seizure.

### DETAILED DESCRIPTION OF THE INVENTION

### Terms

“Administering” an agent can be effected or performed using any of the various methods and delivery systems known to those skilled in the art. The administering can be performed, for example, intravenously, orally, nasally, via cerebrospinal fluid, via implant, transmucosally, transdermally, intramuscularly, and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

“Antibody” shall include, by way of example, both naturally occurring and non-naturally occurring antibodies. Specifically, this term includes polyclonal and monoclonal antibodies, and antigen-binding fragments (e.g., Fab fragments) thereof. Furthermore, this term includes chimeric antibodies (e.g., humanized antibodies) and wholly synthetic antibodies, and antigen-binding fragments thereof.

“Anti-sense nucleic acid” shall mean any nucleic acid which, when introduced into a cell (directly or via expression of another nucleic acid directly introduced into the cell), specifically hybridizes to at least a portion of an mRNA in the cell encoding a protein (i.e., target protein) whose expression is to be inhibited, and thereby inhibits the target protein's expression.

“Catalytic nucleic acid” shall mean a nucleic acid, such as a DNAzyme, that specifically recognizes a distinct substrate and catalyzes the chemical modification of this substrate.

“DNAzyme” shall mean a catalytic nucleic acid that is DNA or whose catalytic component is DNA, and which specifically recognizes and cleaves a distinct target nucleic acid sequence, which can be either DNA or RNA. Each DNAzyme has a catalytic component (also referred to as a “catalytic domain”) and a target sequence-binding component consisting of two binding domains, one on either side of the catalytic domain.

“Inhibiting” neuronal damage shall mean either lessening the likelihood of the damage's onset, or preventing damage entirely. In the preferred embodiment, inhibiting neuronal damage means preventing the damage entirely.

“Nucleic acid” shall mean any nucleic acid molecule, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA).

“Prophylactically effective amount” means an amount sufficient to inhibit the onset of a disorder or a complication associated with a disorder in a subject.

“RAGE” shall mean, without limitation, receptor for advanced glycation endproducts, and can be from human or any other species which produces this protein. The nucleotide and protein (amino acid) sequences for RAGE (both human and murine and bovine) are known. The following references, inter alia, provide these sequences: Schmidt et al, J. Biol. Chem., 267:14987-97, 1992; and Neeper et al, J. Biol. Chem., 267:14998-15004, 1992. Additional RAGE sequences (DNA sequences and translations) are available from GenBank.

“Ribozyme” shall mean a catalytic nucleic acid molecule which is RNA or whose catalytic component is RNA, and which specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

“RNAi” includes, without limitation, a polynucleotide sequence identical or homologous to a target gene (or fragment thereof) linked directly, or indirectly, to a polynucleotide sequence complementary to the sequence of the target gene (or fragment thereof). The RNAi optionally comprises a polynucleotide linker sequence of sufficient length to allow for the two polynucleotide sequences to fold over and hybridize to each other. The linker sequence is designed to separate the antisense and sense strands of RNAi significantly enough to limit the effects of steric hindrances and allow for the formation of a dsRNA molecule, and not to hybridize with sequences within the hybridizing portions of the dsRNA molecule. RNAi is discussed, e.g., in U.S. Pat. No. 6,544,783.

“Specifically inhibit” the expression of a protein shall mean to inhibit that protein's expression (a) more than the expression of any other protein, or (b) more than the expression of all but 10 or fewer other proteins.

“Subject” shall mean any animal, such as a human, non-human primate, mouse, rat, guinea pig or rabbit.

“Therapeutically effective amount” means an amount sufficient to treat a subject afflicted with a disorder or a complication associated with a disorder.

### EMBODIMENTS OF THE INVENTION

This invention provides a method for treating a subject either during or soon after a seizure, in order to reduce the extent of neuronal damage in the subject resulting from the seizure comprising administering to the subject, either during or soon after the seizure, a therapeutically effective amount of an inhibitor of receptor for advanced glycation endproducts (RAGE), so as to thereby reduce the extent of neuronal damage in the subject. In the preferred embodiment, the subject is human.

In one embodiment of the instant method, the neuronal damage comprises cell death in the hippocampus and/or cerebral cortex. In another embodiment of the instant method, the neuronal damage comprises cell dysfunction in the hippocampus and/or cerebral cortex.

In one embodiment of the instant method, the inhibitor is an antibody which, when contacted with RAGE, specifically inhibits binding between RAGE and a ligand thereof. In another embodiment of the instant method, the inhibitor is an anti-sense molecule which specifically inhibits the expression of RAGE in a cell. In another embodiment of the instant method, the inhibitor is an RNAi molecule which specifically inhibits the expression of RAGE in a cell. In still another embodiment of the instant method, the inhibitor is a catalytic nucleic acid which specifically inhibits the expression of RAGE in a cell.

In further embodiments, the inhibitor is administered during the seizure, within three days of the seizure, within one day of the seizure, within six hours of the seizure, within one hour of the seizure or within 20 minutes of the seizure.

This invention further provides a method for inhibiting neuronal damage which would otherwise result from a seizure in a subject predisposed to having a seizure, comprising administering to the subject a prophylactically effective amount of an inhibitor of receptor for advanced glycation endproducts (RAGE), so as to inhibit neuronal damage which would otherwise result from a seizure in the event the subject were to suffer a seizure. In the preferred embodiment, the subject is human.

In one embodiment of the instant method, the neuronal damage comprises cell death in the hippocampus and/or cerebral cortex. In another embodiment of the instant method, the neuronal damage comprises cell dysfunction in the hippocampus and/or cerebral cortex.

In one embodiment of the instant method, the inhibitor is an antibody which, when contacted with RAGE, specifically inhibits binding between RAGE and a ligand thereof. In another embodiment of the instant method, the inhibitor is an anti-sense molecule which specifically inhibits the expression of RAGE in a cell. In another embodiment of the instant method, the inhibitor is an RNAi molecule which specifically inhibits the expression of RAGE in a cell. In still another embodiment of the instant method, the inhibitor is a catalytic nucleic acid which specifically inhibits the expression of RAGE in a cell.

This invention further provides an article of manufacture comprising (a) a packaging material having therein an inhibitor of receptor for advanced glycation endproducts (RAGE) and (b) instructions for using the inhibitor to treat a subject during or soon after a seizure, in order to reduce the extent of neuronal damage in the subject resulting from the seizure.

This invention further provides an article of manufacture comprising (a) a packaging material having therein an inhibitor of receptor for advanced glycation endproducts (RAGE) and (b) instructions for using the inhibitor to inhibit neuronal damage which would otherwise result from a seizure in a subject predisposed to having a seizure.

This invention is illustrated in the Experimental Details section which follows. This section is set forth to aid in an understanding of the invention but is not intended to, and should not be construed to limit in any way the invention as set forth in the claims which follow thereafter.

### EXPERIMENTAL DETAILS

### Introduction

RAGE (Receptor for Advanced Glycation Endproducts) is a member of the immunoglobulin superfamily of cell surface molecules with a diverse repertoire of ligands. Based on its capacity to bind AGEs (advanced glycation endproducts), beta-sheet fibrils, S100/calgranulins and amphoterin, RAGE appears to function as a progression factor promoting pathologic cellular activation in a range of situations. It is hypothesized that RAGE activation promotes seizure-induced cell death following experimentally induced status epilepticus.

### Materials and Methods

Transgenic mice were generated with targeted neuronal overexpression of either wild-type RAGE (Tg wtRAGE) or dominant-negative RAGE, a form lacking the receptor's cytosolic tail (Tg DN-RAGE). Both groups of Tg mice and age- and strain-matched littermate controls were challenged with either systemic kainic acid or pilocarpine. Homozygous RAGE null mice were similarly studied. Acute seizure-induced neuronal damage was examined over the next 1-5 days by silver and FluoroJade staining.

### Results

Both Tg wtRAGE and Tg DN-RAGE displayed prominent upregulation of RAGE. Overexpression these transgenes did not affect seizure severity or seizure-induced mortality in response to either pilocarpine or kainic acid administration. However, following status epilepticus induced by either of these agents, seizure-induced neuronal damage was significantly increased in the CA1 and CA3 hippocampal subfields in Tg wtRAGE (p<0.05), compared with littermate controls. In contrast, damage was strongly reduced in Tg DN-RAGE mice (p<0.05). Consistent with these data, RAGE null mice displayed a 70-80% reduction in cell death in CA1 and CA3 regions, compared with littermate controls (p<0.05).

### Discussion

Following kainic acid- or pilocarpine-induced status epilepticus, RAGE promotes hippocampal neuronal damage. Blockade of RAGE-ligand interaction provides a novel neuroprotective strategy for the prevention of seizure-induced neurotoxicity.

### REFERENCES

1. Hauser, W. A. and Hesdorffer, D.C. N.Y.: Demos, (1990).
2. McNamara, J. O., J. Neurosci. 14(6):3413-3425 (1994).
3. Noebels, J. L., Neuron 16(2):241-244 (1996).
4. Walton, L., Essentials of Neurology. 6th ed Churchill Livingstone Pub, 77-86 (1989).
5. Sackeim, H. A. et al., Arch. Gen. Psych. 44(4):355-360 (1987).
6. Schmidt, A-M., Yan, S-D., Yan, S-F. & Stern, D. M., The multiligand receptor RAGE as a progression factor amplifying immune and inflammatory responses. J. Clin. Invest. 108, 949-955 (2001).
7. Hofmann, M., et al., RAGE mediates a novel proinflammatory axis: the cell surface receptor for S100/calgranulin polypeptides. Cell 97, 889-901 (1999).
8. Taguchi, A., et al., Blockade of RAGE/amphoterin suppresses tumor growth and metastases. Nature 405, 354-360 (2000).
9. Rauvala, H., et al., The adhesive and neurite-promoting molecule p30: analysis of the amino terminal sequence and production of antipeptide antibodies that detect p30 at the surface of neuroblastoma cells and of brain neurons. J. Cell. Biol. 107, 2293-2305 (1987).
10. Hori, O., et al., RAGE is a cellular binding site for amphoterin: mediation of neurite outgrowth and co-expression of RAGE and amphoterin in the developing nervous system. J. Biol. Chem. 270, 25752-25761 (1995).
11. Schafer, B. & Heizmann, C., The S100 family of EF-hand calcium-binding proteins: functions and pathology. TIBS 21, 134-140 (1996).

1. A method for treating a subject either during or soon after a seizure, in order to reduce the extent of neuronal damage in the subject resulting from the seizure comprising administering to the subject, either during or soon after the seizure, a therapeutically effective amount of an inhibitor of receptor for advanced glycation endproducts (RAGE), so as to thereby reduce the extent of neuronal damage in the subject.

2. The method of claim 1, wherein the subject is a human.

3. The method of claim 1, wherein the neuronal damage comprises cell death in the hippocampus and/or cerebral cortex.

4. The method of claim 1, wherein the neuronal damage comprises cell dysfunction in the hippocampus and/or cerebral cortex.

5. The method of claim 1, wherein the inhibitor is an antibody which, when contacted with RAGE, specifically inhibits binding between RAGE and a ligand thereof.

6. The method of claim 1, wherein the inhibitor is an anti-sense molecule which specifically inhibits the expression of RAGE in a cell.

7. The method of claim 1, wherein the inhibitor is an RNAi molecule which specifically inhibits the expression of RAGE in a cell.

8. The method of claim 1, wherein the inhibitor is a catalytic nucleic acid which specifically inhibits the expression of RAGE in a cell.

9. The method of claim 1, wherein the inhibitor is administered during the seizure.

10. The method of claim 1, wherein the inhibitor is administered within three days of the seizure.

11. The method of claim 1, wherein the inhibitor is administered within one day of the seizure.

12. The method of claim 1, wherein the inhibitor is administered within six hours of the seizure.

13. The method of claim 1, wherein the inhibitor is administered within one hour of the seizure.

14. The method of claim 1, wherein the inhibitor is administered within 20 minutes of the seizure.

15. A method for inhibiting neuronal damage which would otherwise result from a seizure in a subject predisposed to having a seizure, comprising administering to the subject a prophylactically effective amount of an inhibitor of receptor for advanced glycation endproducts (RAGE), so as to inhibit neuronal damage which would otherwise result from a seizure in the event the subject were to suffer a seizure.

16. The method of claim 15, wherein the subject is human.

17. The method of claim 15, wherein the neuronal damage comprises cell death in the hippocampus and/or cerebral cortex.

18. The method of claim 15, wherein the neuronal damage comprises cell dysfunction in the hippocampus and/or cerebral cortex.

19. The method of claim 15, wherein the inhibitor is an antibody which, when contacted with RAGE, specifically inhibits binding between RAGE and a ligand thereof.

20. The method of claim 15, wherein the inhibitor is an anti-sense molecule which specifically inhibits the expression of RAGE in a cell.

21. The method of claim 15, wherein the inhibitor is an RNAi molecule which specifically inhibits the expression of RAGE in a cell.

22. The method of claim 15, wherein the inhibitor is a catalytic nucleic acid which specifically inhibits the expression of RAGE in a cell.

23. (canceled)

24. (canceled)

25. An article of manufacture comprising (a) a packaging material having therein an inhibitor of receptor for advanced glycation endproducts (RAGE) and (b) instructions for using the inhibitor to treat a subject during or soon after a seizure, in order to reduce the extent of neuronal damage in the subject resulting from the seizure.

26. An article of manufacture comprising (a) a packaging material having therein an inhibitor of receptor for advanced glycation endproducts (RAGE) and (b) instructions for using the inhibitor to inhibit neuronal damage which would otherwise result from a seizure in a subject predisposed to having a seizure.
